(19) United States
(12) Patent Application Publication
(10) Pub. No.: US 2020/0009130 A1
(43) Pub. Date: Jan. 9, 2020

(54) OCULAR DISTRIBUTION AND PHARMACOKINETICS OF LIFITEGRAST FORMULATIONS

(71) Applicant: SARcode Bioscience Inc., Brisbane, CA (US)

(72) Inventors: Jou-Ku CHUNG, Brookline, MA (US); Elizabeth SPENCER, Madison, WI (US); Matthew HUNT, Columbus, WI (US); Devin WELTY, Watertown, MA (US); Thomas MCCAULEY, Arlington, MA (US)

(73) Assignee: SARcode Bioscience Inc., Brisbane, CA (US)

(21) Appl. No.: 16/470,005

(22) PCT Filed: Dec. 15, 2017
(86) PCT No.: PCT/US17/66653

§ 371 Date: Jun. 14, 2019

Related U.S. Application Data
(60) Provisional application No. 62/435,449, filed on Dec. 16, 2016.

(51) Int. Cl. A61K 31/4725; A61K 9/00; A61K 47/02; A61K 47/18

(52) U.S. Cl. CPC A61K 31/4725; A61K 9/0048; A61K 9/08; A61K 47/183; A61K 47/02

(57) ABSTRACT

The present invention provides lifitegrast formulations useful for the treatment of immune-related diseases of the ocular surface. The formulations and methods provided herein are particularly useful for treatment of ocular anterior segment tissues.

### RELATED APPLICATIONS

This application claims priority to U.S. Provisional Patent Application No. 62/435,449, filed Dec. 16, 2016, which is hereby incorporated by reference in its entirety.

### FIELD OF THE INVENTION

The present invention provides lifitegrast formulations useful for the treatment of immune-related diseases of the ocular surface (e.g., dry eye disease). The formulations and methods provided herein are particularly useful for treatment of ocular anterior segment tissues, in particular, the conjunctiva and cornea.

### BACKGROUND OF THE INVENTION

Dry eye disease (DED) is an ocular disorder associated with surface tissue damage and impaired tear production and is commonly encountered in clinical practice (Ocul. Surf., 2007, 5:93-107). Although the etiology of DED is complex, there is strong evidence that supports chronic inflammation as a significant factor in the pathogenesis of DED (Invest. Ophthalmol. Vis. Sci. 2000, 41:1356-1363; Invest. Ophthalmol. Vis. Sci., 2012, 53:5443-5450; Ocul. Surf. 2005, 3:S161-S164). Several studies have shown that inflammatory mediators can be found in the ocular surface tissues of patients with DED, specifically in the corneal and conjunctival epithelium (Ocul. Surf. 2005, 3:S161-S164; Arch. Ophthalmol. 2006, 124:710-716; Am. J. Ophthalmol. 2009, 147:198-205).

Lifitegrast (Xiidra™) is a novel small molecule lymphocyte function-associated antigen 1 (LFA-1) antagonist that has recently been approved by the US Food and Drug Administration for the treatment of signs and symptoms of DED (FDA approves new medication for dry eye disease. Silver Spring, Md.: US Food and Drug Administration; Jul. 12, 2016. http://www.fda.gov/newsevents/newsroom/pressannouncements/ucm510720.htm; Accessed Jul. 12, 2016). The current understanding of the mechanism of action of lifitegrast is that it decreases T cell-mediated inflammation associated with DED by blocking the interaction between the integrin LFA-1 and intercellular adhesion molecule 1 (ICAM-1), thereby preventing inflammatory cell activation and migration (Pflugfelder, S. C. et al. , J. Ocul. Pharmacol. Ther. doi:10.1089/jop.2016.0105; Perez V. L. et al., Ocul. Surf 2016, 14:207-215). Lifitegrast is administered as a 5.0% ophthalmic solution applied to each eye twice daily (b.i.d.; ˜12 hours apart).

There is an ongoing need for ophthalmic formulations having advantageous pharmacokinetic properties and ocular tissue distribution. The compositions and methods described herein are directed to these and other ends.

### SUMMARY OF THE INVENTION

The present invention provides, inter alia, a method of treating an immune-related disease of the ocular surface in a subject. In some embodiments, the method of the invention comprises topically administering to the eye of the subject an effective amount of lifitegrast, or a pharmaceutically acceptable salt thereof, in a formulation that provides a lifitegrast maximum concentration (Cmax) of greater than about 5190 ng/mL in an ocular anterior segment tissue of the eye for a 1.75 mg dose of lifitegrast.

The method is particularly useful for treating chronic inflammation-related diseases, such as dry eye disease (DED) and advantageously targets the anterior segment tissue, including the conjunctiva (palpebral/bulbar), cornea, and/or sclera (anterior) segment tissue of the eye.

The formulation can achieve a lifitegrast Cmax in the ocular anterior segment tissue of the eye within about 0.25 to about 1 hours. The lifitegrast Cmax can be in the range of about 5190 to about 14200 ng/mL in the ocular anterior segment tissue of the eye (e.g., a lifitegrast Cmax of greater than or equal to about 9620 ng/mL in the conjunctiva (palpebral), about 5190 ng/mL in the cornea, about 5870 ng/mL in the sclera (anterior), and about 9370 ng/mL in the conjunctiva (bulbar)). Low levels of lifitegrast (e.g., less than or equal to about 826 ng/mL) are found in the posterior segment tissue of the eye. The method includes administering the formulation twice daily and in intervals of about 12 hours apart.

The present invention further provides an ophthalmic formulation comprising lifitegrast, or a pharmaceutically acceptable salt thereof, wherein after topically administering the formulation to an eye of a subject, a lifitegrast maximum concentration (Cmax) of greater than about 5190 ng/mL is provided in an ocular anterior segment tissue of the eye for a 1.75 mg dose of lifitegrast.

The pharmaceutically acceptable salt includes a sodium salt. The ophthalmic formulation can include lifitegrast at various concentrations (e.g., at 5.0% by weight) and may include other excipients such as sodium chloride, sodium phosphate dibasic anhydrous, sodium bicarbonate, ethylenediaminetetraacetic acid (EDTA), and sodium thiosulfate pentahydrate. The formulation may be prepared at a useful pH, e.g., pH of about 6.9 to about 7.35.

### BRIEF DESCRIPTION OF THE DRAWING

FIG. 1 is a plot of mean (standard deviation [SD]) concentration of lifitegrast in female pigmented rabbit ocular tissues and plasma at day 5. Formulation #1: anterior segment tissues (A), posterior segment tissues and plasma (B); formulation #2: anterior segment tissues (C), posterior segment tissues and plasma (D). Data excludes lens concentration, for which concentrations were <10 ng/g for both formulations.

### DETAILED DESCRIPTION

As described herein, the inventors have discovered, after extensive investigation, ophthalmic formulations and methods particularly well suited for topical administration of lifitegrast for ophthalmic use. The formulations are stable, well tolerated, and capable of delivering therapeutically effective amounts of lifitegrast to target sites, including sites on the surface of and/or within the eye. Surprisingly, the formulations provide lifitegrast localized in anterior ocular segment tissues, in particular the conjunctiva and cornea, with low concentrations in the posterior segment tissues and plasma. The data and disclosure provided here suggest that lifitegrast can be formulated to reach advantageous ocular surface tissues for ophthalmic use (e.g., DED treatment), while having limited potential for off-target systemic or ocular effects.

The invention will be described in greater detail by way of specific examples. The following examples are offered for illustrative purposes, and are not intended to limit the invention in any manner. Those of skill in the art will readily recognize a variety of noncritical parameters which can be changed or modified to yield essentially the same results.

### EXAMPLE 1

### Ocular Distribution and Pharmacokinetics of Lifitegrast Following Repeat Ocular Dose Administration in Pigmented Rabbits

In this experiment, female pigmented rabbits were assigned to receive one of two formulations of lifitegrast (#1 and #2) for 5 consecutive days. Each treatment group consisted of 25 rabbits. Animals received a single topical ocular dose of lifitegrast in each eye b.i.d.

(except on study day 5), approximately 12 hours apart (±1 hour), at a target dose level of 1.75 mg/eye/dose (35 μL/eye/dose). On study day 5, animals were dosed in the morning only. Treatment groups 1 and 2 were dosed on separate days. The in-life portion of the study was conducted in May 2014. This study adhered to the ARVO Statement for the Use of Animals in Ophthalmic and Vision Research and all procedures in the study were in compliance with the Animal Welfare Act Regulations (9 CFR 3).

### Test Animals

Fifty female New Zealand Red/White F1 Cross rabbits from Covance Research Products (Denver, Pa., USA) were used in the study. The animals were acclimated to study conditions for 13 days before dose administration. At dosing, the animals weighed 3177 to 4271 g and were at least 6 months of age. Animals were housed individually, in suspended stainless steel cages, with food and water ad libitum, and under a 12-hour light/12-hour darkness cycle throughout the study. Animals were not randomized, but were assigned to animal numbers based on overall health and the results of predose ophthalmic examinations. Ophthalmic examinations were performed by a board-certified veterinary ophthalmologist once at baseline (pre dose) using a Kowa hand-held slit-lamp biomicroscope and an indirect ophthalmoscope with a condensing lens to ensure the study animals were healthy and had no ocular findings pre-treatment.

### Dose Preparation and Formulation

Formulations for the study that would be representative of the clinical material (confirmed by analytical data) were prepared at Covance Laboratories Inc. In each formulation, lifitegrast was added to dose vehicle while stirring, and pH adjusted with hydrochloric acid (HCl) and/or sodium hydroxide (NaOH). The formulation was stirred until a clear solution was obtained, filtered with a 0.22 μm filter (Millipore® Millex-GV, 0.22 μm, Durapore® PVDF) and stored at approximately 5° C. Analysis of the formulations was performed by Almac Sciences, Souderton, Pa., USA, and concentrations were measured as 49.4 and 49.3 mg/mL for formulations #1 and #2, respectively.

Formulation #1: dose vehicle consisted of sterile water for injection, sodium chloride, sodium phosphate dibasic anhydrous, sodium bicarbonate, ethylenediaminetetraacetic acid (EDTA), and sodium thiosulfate pentahydrate, adjusted to a pH of 7.30 with HCl. After addition of lifitegrast (50 mg/mL), the pH was adjusted to 6.90.

Formulation #2: dose vehicle consisted of sterile water for injection, sodium chloride, sodium phosphate dibasic anhydrous, and sodium thiosulfate pentahydrate. After addition of lifitegrast (50 mg/mL), the pH of the formulation was adjusted to 7.35.

### Dose Administration

Topical ocular doses of lifitegrast (35 μL/eye/dose) were administered into the cul-de-sac of the eye via a calibrated positive displacement micropipette to ensure contact with the conjunctiva. The right eye was dosed first; collection times (below) were based on the time of dosing of the second (left) eye. Animals were not fasted before dose administration.

### Tissue Collection

Animals were euthanized with sodium pentobarbital and blood and ocular tissues were collected from 5 animals per group per time point at 0.25, 0.5, 1, 3, and 8 hours post last dose on study day 5. Blood (˜5 mL) was collected into tubes containing tripotassium ethylenediaminetetraacetic acid (K₃EDTA), an anticoagulant. Blood was centrifuged to obtain plasma. The following ocular tissues were collected using a frozen collection technique: aqueous humor, conjunctiva (bulbar and palpebral), choroid-retinal pigment epithelium (choroid-RPE), cornea, iris-ciliary body, lens, optic nerve, retina, sclera (anterior and posterior), and vitreous humor. Plasma and ocular tissues were stored at approximately −70° C.

### Liquid Chromatography Tandem Mass Spectroscopy Analysis

Lifitegrast concentrations were measured by a validated liquid chromatography tandem mass spectrometry (LC-MS/MS) method in rabbit plasma and multiple eye matrices (analyses were performed at ICON Development Solutions, LLC, Whitesboro, N.Y., USA). The method was qualified for the analysis of rabbit aqueous and vitreous humor, using rabbit plasma as a proxy matrix. The LC-MS/MS analysis was performed using a Sciex API-5000™ mass spectrometer (SCIEX, Framingham, Mass., USA) coupled with a Shimadzu high-performance liquid chromatography (HPLC) system. The chromatographic separation was achieved on a Waters SymmetryShield™ RP18 HPLC column, 2.1×50 mm, 3.5 μm (Waters Corporation, Milford, Mass., USA), with a mobile phase gradient. The mass spectrometer was operated in turbo ionspray (positive ion) mode and the resolution setting used was unit for both quadrupoles Q1 and Q2. The lowest level of quantification for lifitegrast was 0.500 ng/tissue (the standard curve range was 0.5-100 ng/sample).

### Data Analysis

Non-compartmental analysis (Gibaldi M, Perrier D. Pharmacokinetics. 2nd ed. New York, N.Y.: Dekker; 1982) was applied to the mean tissue and plasma lifitegrast concentration data. PK analyses included, wherever possible, determination of maximum concentration (Cₘₐₓ) in ocular tissues and plasma, time to Cₘₐₓ (tₘₐₓ), area under the concentration-time curve (AUC) from time 0 to the last measurable time point (AUG₀₋ₜ), and elimination phase half-life (t_{1/2}). PK analysis was performed using Phoenix® WinNonlin® (Version 6.2; Pharsight Corporation, Sunnyvale, Calif., USA). Nominal doses and sampling times were used. Concentration values below the lower limit of quantification (BLQ; <0.500 ng/mL or <0.500 ng/sample, as appropriate) were treated as zero. If two-thirds of the samples were BLQ at a given time point, the mean was reported as “not calculated” in descriptive statistics and treated as zero in the PK analysis.

### Results

All animals (n=50) had normal ophthalmic examinations pre dose. For animals receiving formulation #1 (n=25), mean (standard deviation [SD]) body weight was 3710 (260) g and mean (SD) dose was 0.935 (0.0632) mg/kg. For animals receiving formulation #2 (n=25), mean (SD) body weight was 3610 (242) g and mean (SD) dose was 0.959 (0.0658) mg/kg. Both formulations were well tolerated and no clinical safety observations were made during the study period.

The PK parameters of lifitegrast for ocular tissues and plasma for each formulation are summarized in Table 1. Cₘₐₓ and AUG₀₋₈ (AUC from 0 to 8 hours) values for the plasma and ocular tissues were generally similar between formulations #1 and #2. Exposure of lifitegrast (assessed by AUC₀₋₈) following administration of either formulation was highest in the conjunctiva (palpebral), followed by the cornea, sclera (anterior), conjunctiva (bulbar), sclera (posterior), iris-ciliary body, aqueous humor, and choroid-RPE in order of decreasing magnitude. Concentrations of lifitegrast were highest in the anterior ocular surface tissues, with Cₘₐₓ for the conjunctiva (palpebral and bulbar) and cornea ranging from 5930 to 14200 ng/g for formulation #1 and from 5190 to 9620 ng/g for formulation #2. AUG₀₋₈ for these tissues ranged from 13400 to 30800 ng.h/g and 12000 to 36600 ng.h/g, for formulations #1 and #2, respectively. The Cₘₐₓ of lifitegrast in the iris-ciliary body, aqueous humor, and choroid-RPE ranged from 79 to 190 ng/g for formulation #1 and from 45.9 to 195 ng/g for formulation #2. AUC₀₋₈ in these tissues was 530 to 1130 ng.h/g for formulation #1 and 231 to 778 ng.h/g for formulation #2.

For the sclera, exposure to lifitegrast was significantly lower in the posterior tissue versus the anterior tissue. The Cₘₐₓ of lifitegrast was 11200 and 5870 ng/g in the anterior sclera and 826 and 369 ng/g in the posterior sclera for formulations #1 and #2, respectively. The AUC₀₋₈ of lifitegrast was 17500 and 11200 ng.h/g in the anterior sclera and 2360 and 1570 ng.h/g in the posterior sclera for formulations #1 and #2, respectively. Limited measurable concentrations of lifitegrast were observed in the optic nerve, retina, and vitreous humor for both formulations. The mean Cₘₐₓ in these tissues ranged from BLQ to 36 ng/g, which was significantly lower than those observed in anterior ocular tissues. AUC₀₋₈ in the optic nerve, retina, and vitreous humor could not be calculated due to insufficient measurable data, which suggested that distribution to the back of the eye was very limited.

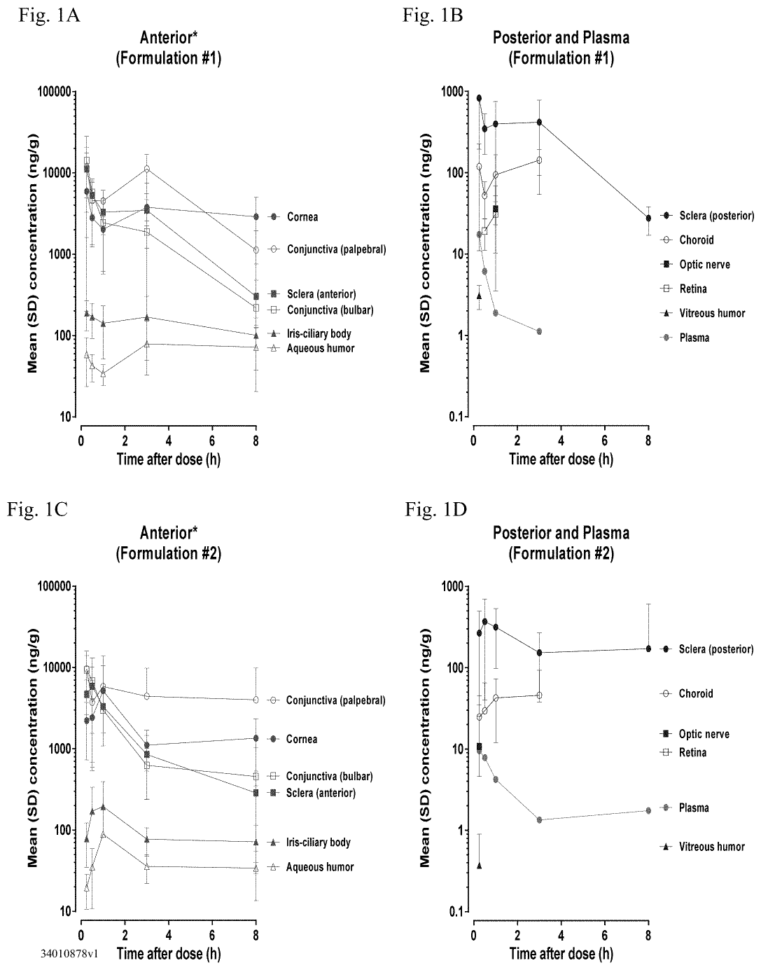

Concentrations were also very low in the lens, with Cₘₐₓ=3.85 ng/g and AUC₀₋₈=5.44 ng.h/g for formulation #1, and Cₘₐₓ=0.794 ng/g (AUC₀₋₈ not calculated) for formulation #2. Across all tissues, tₘₐₓ was generally between 0.25 and 1 hours, indicating rapid absorption following topical ocular administration. Due to the lack of a distinct elimination phase, estimation of t_{1/2} in most ocular tissues could not be calculated, but in the conjunctiva (bulbar), t_{1/2} was 2.02 hours (formulation #1), and in the sclera, (anterior) t_{1/2} was 1.97 and 2.32 hours for formulations #1 and #2, respectively. The mean concentration of lifitegrast in the anterior (FIGS. A, C; excluding lens) and posterior (FIGS. B, D) ocular segment tissues over the 8 hours post dose at day 5 is shown in FIG. 1.

Low plasma levels of lifitegrast were observed (Cₘₐₓ values of 17.4 and 9.52 ng/mL, AUC₀₋₈ values of 11.2 and 16.4 ng.h/g, for formulations #1 and #2, respectively), following five b.i.d. doses. After a topical ocular dose of formulation #1, maximum plasma concentrations of lifitegrast were reached within 0.25 hours (tₘₐₓ) and declined with a plasma t_{1/2} value of 0.850 hours. Due to the lack of a distinct elimination phase, t_{1/2} in plasma for formulation #2 could not be calculated (FIGS. B, D).

TABLE 1
Pharmacokinetic Parameters of Lifitegrast Calculated for
Various Ocular Tissues and Plasma for Each Formulation

| | Formulation #1 | | | Formulation #2 | | |
|---|---|---|---|---|---|---|
| | Cₘₐₓ | | AUC₀₋₈ | Cₘₐₓ | | AUC₀₋₈ |
| | (ng/mL | tₘₐₓ | (ng · h/mL | (ng/mL | tₘₐₓ | (ng · h/mL |
| Tissue | or ng/g) | (h) | or ng · h/g) | or ng/g) | (h) | or ng · h/g) |
| Conjunctiva (palpebral) | 11900 | 0.250 | 30800 | 9620 | 0.250 | 36600 |
| Cornea | 5930 | 0.250 | 25500 | 5190 | 1.00 | 15200 |
| Sclera (anterior) | 11200 | 0.250 | 17500 | 5870 | 0.500 | 11200 |
| Conjunctiva (bulbar) | 14200 | 0.250 | 13400 | 9370 | 0.250 | 12000 |
| Sclera (posterior) | 826 | 0.250 | 2360 | 369 | 0.500 | 1570 |
| Iris-ciliary body | 190 | 0.250 | 1130 | 195 | 1.00 | 778 |
| Aqueous humor | 79.0 | 3.00 | 530 | 89.5 | 1.00 | 340 |
| Choroid-RPE | 119 | 0.250 | 492 | 45.9 | 3.00 | 231 |
| Plasma | 17.4 | 0.250 | 11.2 | 9.52 | 0.250 | 16.4 |
| Lens | 3.85 | 1.00 | 5.44 | 0.794 | 3.00 | NR |
| Optic nerve | 36.0 | 1.00 | NR | 10.8 | 0.250 | NR |
| Retina | 31.2 | 1.00 | NR | NR | NR | NR |
| Vitreous humor | 2.09 | 0.250 | NR | 0.372 | 0.250 | NR |

AUC₀₋₈, area under the concentration-time curve from 0 to 8 hours; choroid-RPE, choroid-retinal pigment epithelium; Cₘₐₓ, maximum concentration; NR = not reported due to limited measurable data; tₘₐₓ, time to maximum concentration.

Based on the above data, it was observed that following twice daily ocular administration of lifitegrast for 5 days in pigmented rabbits, distribution and exposure of lifitegrast was generally highest in the anterior ocular segment tissues, in particular the conjunctiva and cornea, while low concentrations of lifitegrast were observed in the posterior segment tissues. Lifitegrast is indicated for the treatment of DED, an ocular surface disorder in which T-cell infiltration and inflammation have been observed in the conjunctiva and cornea (Ocul. Surf 2005;3:S161-S164; Arch. Ophthalmol. 2006;124:710-716; Am. J. Ophthalmol. 2009;147:198-205; Stem M. E. et al., Invest. Ophthalmol. Vis. Sci. 2002;43:2609-2614). Thus, the predominant distribution of lifitegrast in these tissues corresponds well to the intended site of action, supported by clinical effects on signs and symptoms of dry eye (Holland E. J. et al., Curr. Med. Res. Opin. 2016;32:1759-1765; Holland E. J., et al., Ophthalmology. doi:10.1016/j.ophtha.2016.09.025).

The distribution profile further indicates low potential for off-target effects in the posterior ocular segment tissues. Plasma concentrations of lifitegrast were also notably low, as observed previously in a phase I study in healthy volunteers (Semba C. P. et al., J. Ocul. Pharmacol. Ther. 2011;27:99-104) and a subpopulation of the 1-year safety study SONATA (Donnenfeld E. D., et al., Cornea. 2016;35:741-748, and plasma t_{1/2} was short (0.850 hours). These data indicate limited potential for systemic side effects with lifitegrast. Consistent with these observations, the safety profile of lifitegrast in clinical trials has shown the drug to be generally well tolerated, with no suggestion of systemic toxicities (Sheppard J. D. el al. Ophthalmology 2014;121:475-483; Tauber J. et al., Ophthalmology; 2015;122:2423-2431; Holland E. J., et al., Ophthalmologydoi: 10.1016j.ophtha.2016.09.025). An additional point of note is that the ocular tissue elimination half-life (t_{1/2}) determined in this study (e.g., ˜2 hours for the conjunctiva bulbar) supports the approved twice daily dosing of lifitegrast (FDA approves new medication for dry eye disease. Silver Spring, Md.: US Food and Drug Administration; Jul. 12, 2016. http://www.fda.gov/newsevents/newsroom/pressannouncements/ucm510720.htm. Accessed Jul. 12, 2016). Because the dosing interval is long relative to the time needed to eliminate the drug from the ocular tissues, there is limited potential for drug accumulation. The distribution and exposure of lifitegrast in the plasma and ocular tissues were comparable between formulations, and both formulations were well tolerated with no clinically relevant safety observations.

The rabbit is the most common species used for evaluating ocular distribution because the rabbit eye is large enough to perform topical drug deliveries (Short B. G., et al., Toxicol. Pathol. 2008;36:49-62) and comparable in size with a human eye. Pigmented rabbits of the crossed strain New Zealand Red/White F1 were used to increase comparability with humans by accounting for the potential for melanin to affect the distribution of the drug (Durairaj C. et al., Exp Eye Res. 2012;98:23-27). Our study found relatively low lifitegrast concentrations in the iris-ciliary body relative to the conjunctiva and cornea, indicating that lifitegrast has relatively low potential for melanin binding.

Previous investigational ocular PK studies of lifitegrast have been carried out in rats (Rao V. R. et al., Invest Ophthalmol Vis Sci. 2010;51:5198-5204) and dogs (Murphy C J, et al., Invest Ophthalmol Vis Sci. 2011;52:3174-3180) using radiolabeling. Consistent with our study, concentrations of radioactivity were found to be the highest in the anterior segment tissues (bulbar conjunctiva, palpebral conjunctiva, and cornea) in dogs, 30 minutes after topical administration (Murphy C J, et al., Invest Ophthalmol Vis Sci. 2011;52:3174-3180). Similarly in rats, postdose radioactivity at 0.5 hours was highest in the conjunctiva and cornea (Rao V. R. et al., Invest Ophthalmol Vis Sci. 2010;51:5198-5204). Contrary to our findings. Rao et al (Rao V. R. et al., Invest Ophthalmol Vis Sci. 2010;51:5198-5204) observed comparable radioactivity in the cornea versus the iris-ciliary body. Although uncertain, a putative explanation is the difference in ocular anatomy between rodents and rabbits, including anterior chamber depth (Vezina M. et al Assessing Ocular Toxicology in Laboratory Animals, Molecular and Integrative Toxicology. New York, N.Y.: Humana Press; 2013:1-21).

Various modifications of the invention, in addition to those described herein, will be apparent to those skilled in the art from the foregoing description. Such modifications are also intended to fall within the scope of the appended claims. Each reference cited in the present application, including all patents, patent applications, and non-patent literature, is incorporated herein by reference in its entirety.

What is claimed is:

1. A method of treating an immune-related disease of the ocular surface in a subject, the method comprising topically administering to the eye of the subject an effective amount of lifitegrast, or a pharmaceutically acceptable salt thereof, in a formulation that provides a lifitegrast maximum concentration (Cmax) of greater than about 5190 ng/mL in an ocular anterior segment tissue of the eye for a 1.75 mg dose of lifitegrast.

2. The method of claim 1, wherein the immune-related disorder is dry eye disease (DED).

3. The method of claim 1, wherein the anterior segment tissue comprises the conjunctiva (palpebral/bulbar), cornea, and/or sclera (anterior) segment tissue of the eye.

4. The method of claim 1, wherein lifitegrast provides a Cmax in the ocular anterior segment tissue of the eye within about 0.25 to about 1 hours.

5. The method of claim 1, wherein the lifitegrast Cmax is in the range of about 5190 to about 14200 ng/mL in the ocular anterior segment tissue of the eye.

6. The method of claim 1, wherein the formulation provides a lifitegrast Cmax of greater than or equal to about 9620 ng/mL in the conjunctiva (palpebral).

7. The method of claim 1, wherein the formulation provides a lifitegrast Cmax of greater than or equal to about 5190 ng/mL in the cornea.

8. The method of claim 1, wherein the formulation provides a lifitegrast Cmax of greater than or equal to about 5870 ng/mL in the sclera (anterior).

9. The method of claim 1, wherein the formulation provides a lifitegrast Cmax of greater than or equal to about 9370 ng/mL in the conjunctiva (bulbar).

10. The method of claim 1, wherein the formulation provides a lifitegrast Cmax of less than or equal to about 826 ng/mL in the posterior segment tissue of the eye.

11. The method of claim 10, wherein the posterior segment tissue is sclera (posterior) tissue.

12. The method of claim 1, wherein the Cmax is provided in the eye of a rabbit.

13. The method of claim 1, wherein the method comprises administering the formulation twice daily.

14. The method of claim 1, wherein the method comprises administering the formulation in intervals of about 12 hours apart.

15. An ophthalmic formulation comprising lifitegrast, or a pharmaceutically acceptable salt thereof, wherein after topically administering to an eye of a subject, the formulation provides a lifitegrast maximum concentration (Cmax) of greater than about 5190 ng/mL in an ocular anterior segment tissue of the eye for a 1.75 mg dose of lifitegrast.

16. The ophthalmic formulation of claim 15, wherein the pharmaceutically acceptable salt is a sodium salt.

17. The ophthalmic formulation of claim 15, wherein the formulation comprises 5% by weight of lifitegrast.

18. The ophthalmic formulation of claim 15, comprising thiosulfate pentahydrate.

19. The ophthalmic formulation of claim 15, comprising ethylenediaminetetraacetic acid (EDTA).

20. The ophthalmic formulation of claim 15, comprising sodium chloride, sodium phosphate dibasic anhydrous, sodium bicarbonate, ethylenediaminetetraacetic acid (EDTA), and sodium thiosulfate pentahydrate.

21. The ophthalmic formulation of claim 15, having a pH of 6.9.

22. The ophthalmic formulation of claim 15, having a pH of 7.35.
